(19) United States
(12) Patent Application Publication
(10) Pub. No.: US 2007/0173866 A1
(43) Pub. Date: Jul. 26, 2007

(54) Surgical hemostatic clip

(75) Inventors: Greg Sorrentino, Wallingford, CT (US); Kenneth H. Whitfield, New Haven, CT (US)

Correspondence Address: Michael D. Switzer;U.S. Surgical,, a division of TYCO Healthcare Group, LP, 150 Glover Avenue, Norwalk, CT, 06856, US

(73) Assignee: Tyco Healthcare Group, LP

(21) Appl. No.: 11/338,911

(22) Filed: Jan. 23, 2006

(51) Int. Cl. A61B 17/08

(52) U.S. Cl. 606/151

(57) ABSTRACT

A surgical clip for a clip applier has a first leg connected to a second leg at a first location and the first leg separated from the second leg by a predetermined distance at a second location spaced from the first location. The first leg has a first distal end and the second leg has a second distal end. The first leg has a tissue gripping region and the second leg has the second tissue gripping region. The first tissue gripping region has a first recess on the first leg. The second tissue gripping region has a second recess on the second leg. When the clip is compressed the first recess is compressed toward the second recess and the first recess and the second recess overlap to form a two dimensional polygonal pattern with one another.

### BACKGROUND

1. Technical Field

This invention relates to surgical clips, and more particularly, to hemostatic surgical clips for application to blood vessels or body tissue.

2. Background

Ligation or occlusion of veins, arteries or blood vessels is a critical part of some surgical procedures. A severed vessel requires closure on both sides of a severance site before actual cutting takes place using another cutting instrument or knife.

Surgeons have used thread or suture material to tie a vessel prior to severing the vessel. This procedure was often time-consuming and required great surgeon dexterity. In many instances, the assistance of a nurse or attending surgeon was necessary to perform this procedure to perfect grasping and tying the vessel, then repeatedly testing the vessel to ensure closure. If complete closure of the vessel was not achieved using the suture material, then the sequence was repeated.

Surgical clips and hemostatic surgical clip appliers greatly enhances the art of vessel occlusion. Surgical clips are now commonly used for vessel ligation and occlusion. Examples of surgical hemostatic clips are described in U.S. Pat. Nos. 5,501,693, 5,171,253; 5,171,252; 5,100,420; 5,084,057; 4,971,198; 4,844,066; 4,799,481; 4,702,247; 4,414,721; 4,188,953; 4,146,130; 3,867,944; and 3,363,628, which are all herein incorporated by reference in their entirety.

Certain factors are important to the performance of a surgical hemostatic clip and to achieve proper tissue exudation and occlusion. The clip should not slip or become dislodged from a vessel after it has been applied. If the clip is not securely positioned, blood or other bodily fluid may begin flowing into the surgical site through the unclamped vessel. A surgeon must locate and reclamp the vessel. Depending upon the type and location of the surgery, reclamping the vessel may be difficult, and reduce an overall productivity of the procedure. A clip should fully and completely close about a vein, artery, vessel or other conduit and completely stop the flow of blood or fluid therethrough. A clip that does not completely occlude the blood or fluid flow may have to be removed thus requiring application of a second clip.

Some surgical hemostatic clips are U-shaped or V-shaped. These clips have a pair of legs joined at one end by an apex or crown and spaced apart at the opposed ends to define a gap between the legs. The desired vessel is introduced in the gap and the legs are compressed. The clip thus occludes the vessel using the legs.

The legs have surfaces that contact tissue. These “tissue gripping surfaces” of the hemostatic clip can be made in a manner to improve the occluding functions of the hemostatic clip. The surfaces can also restrict dislocation of the hemostatic clip after it has been applied to the target blood vessel. However, often legs have a relatively small tissue gripping surface. Care must be taken when designing such tissue gripping surfaces to ensure that the most productive use of the relatively small tissue gripping surface is made to accomplish the occlusion. A significant aspect of the tissue gripping surfaces is this retention of the hemostatic clip on the tissue. Accordingly, there is a need in the art for an improved surgical hemostatic clip to provide an optimum vessel occlusion and optimal clip retention on tissue during a surgical procedure.

### SUMMARY

According to a first aspect thereof, there is provided a surgical clip for a clip applier. The surgical clip has a first leg connected to a second leg at a first location. The first leg is separated from the second leg by a predetermined distance at a second location spaced from the first location. The first leg has a first distal end and the second leg has a second distal end. The first leg has a tissue gripping region and the second leg has the second tissue gripping region. The first tissue griping region has a first recess on the first leg, and the second tissue gripping region has a second recess on the second leg. When the clip is compressed the first recess is compressed toward the second recess. The first recess and the second recess overlap to form a two dimensional polygonal pattern with one another.

According to another aspect of the present disclosure the first leg has a plurality of first recesses and the second leg has a plurality of second recesses with substantially the same orientation. Each first recess registers with another of the second recesses when the first clip leg is compressed toward the second clip leg.

According to another aspect of the present disclosure, the first recess is compressed toward the second recess and the first and the second recess form a two dimensional diamond shaped pattern.

According to yet another aspect of the present disclosure, the first recess has an apex portion and a first and a second distal leg portion extending from the apex portion.

According to a further aspect, the surgical clip has the second recess with an apex portion and a first and a second distal leg extending from the apex portion.

According to another aspect of the present disclosure, the first clip leg is compressed to the second clip leg and the apex portion of the first clip leg and the apex portion of the second leg point toward opposite directions.

According to yet another aspect of the present disclosure, the first leg has the plurality of first recesses. Each of the first recesses has an apex portion and a first and a second leg extending from the apex portion with the apex portions of each of the recesses pointing toward the same direction.

According to another aspect of the present disclosure, the second leg has the plurality of second recesses with each of the recesses having an apex portion and a first and a second leg extending from the apex portion. The apex portions of the second recesses point toward an opposite direction relative to the apex portion of the first recesses.

According to another aspect of the present disclosure, each recess is a substantially “V” shaped recess.

According to still another aspect of the present disclosure, the clip has a longitudinal channel. The channel extends through each of the recesses of at least the first leg and the second leg.

According to another aspect of the present disclosure, the clip has a grip feature on an outer clip leg surface.

According to yet another aspect of the present disclosure, each of the recesses does not contact another of the recesses.

According to another aspect of the present disclosure, the surgical clip is a hemostatic clip and is made from a material selected from the group consisting of stainless steel, a polymer, titanium, a biocompatible material, and any combinations thereof.

In another example of the present disclosure, the surgical clip has a first leg connected to a second leg at a first location. The first leg is separated from the second leg by a predetermined distance at a second location spaced from the first location. The first leg has a first distal end and the second leg has a second distal end. The clip also has a first gripping pattern on the first leg with a plurality of first recesses and a second gripping pattern on the second leg having the plurality of second recesses. The first recesses are in registration with the second recesses when the first leg is compressed towards the second leg such that each of the first recesses registers with the second recesses to form a two dimensional polygonal pattern. The clip also has the first leg with a channel extending through each of the recesses on the first leg and the second leg has a second channel extending through each of the recesses on the second leg. According to another aspect thereof each recess is “V” shaped.

According to another aspect of the present disclosure, the surgical clip has both the first channel and the second channel unconnected with one another or with the first channel and the second channel connected to one another. The clip may also have each recess being “U” shaped.

According to another aspect of the present disclosure, when each of the first recesses overlap and align with the second recesses when the clip is compressed the first recesses and the second recesses together form a two dimensional diamond shaped pattern with one another.

According to yet another aspect of the present disclosure there is provided a surgical clip for a clip applier. The surgical clip has a first leg connected to a second leg at a first location and the first leg separated from the second leg by a predetermined distance at a second location spaced from the first location. The first leg has a first distal end and the second leg has a second distal end. The first leg has a tissue gripping region and the second leg having the tissue gripping region. The first tissue griping region has a plurality of first “V” shaped recesses on the first leg with each recess having a first apex and the second tissue gripping region has a plurality of second “V” shaped recess on the second leg with each recess having a second apex. When compressed the first “V” shaped recesses on the first leg align and overlap with the second “V” shaped recesses on the second leg. When the clip is compressed each apex of the recesses points in an opposite direction.

According to yet a further aspect of the present disclosure there is provided a method of treating a hemostatic clip. The method has the steps of heating the clip in a vacuum to a desired temperature. The temperature is in a range of 1,275 degrees Fahrenheit. The method has the steps of holding the clip at the desired temperature for a desired period of time and soaking the clip in an inert gas for a desired period of time. The method has the steps of exposing the clip in a gas and cooling the clip to room temperature at a slow cooling rate.

According to yet a further aspect of the present disclosure there is provided a method of treating a hemostatic clip where the method has the step of soaking the clip in an argon gas.

According to yet a further aspect of the present disclosure, the method has the step of the clip being exposed to argon gas.

According to yet a further aspect of the present disclosure, the clip is a biocompatible titanium and the clip is soaked for about one hour.

According to yet a further aspect of the present disclosure there is provided a method of annealing a hemostatic clip comprising heating the clip in a vacuum to a desired temperature, wherein the temperature is in a range that includes 1,250 degrees Fahrenheit to 1,275 degrees Fahrenheit and holding the clip in the vacuum at the desired temperature for a desired period of time. The method also has the steps of soaking the clip for a desired period of time in the inert gas with the period of time being about one hour and exposing the clip in an argon gas to control a microstructure of the clip and slow cooling the clip to ambient temperature. The clip is biocompatible titanium.

According to yet a further aspect of the present disclosure the method has the step of machining a first plurality of recesses having a first apex in a first leg of the clip, and machining a second plurality of recesses having a second apex in a second leg of the clip. When the clip is compressed the second plurality of recesses are in registration with the first plurality of recesses. The first apex is disposed one hundred and eighty degrees from the second apex to form a compressed diamond shaped pattern.

According to another aspect of the present disclosure, there is provided a surgical clip for a clip applier. The surgical clip has a first leg connected to a second leg at a first location and the first leg is separated from the second leg by a predetermined distance at a second location opposite the first location. The first leg forms a first distal end. The second leg forms a second distal end. The clip also has a plurality of recesses on the first leg with the plurality of recesses each having a first leading edge. The clip also has a plurality of the recesses on the second leg. The recesses on the second leg have a second leading edge. The plurality of first recesses are in registration with the second recesses when the first leg is compressed to the second leg. The first leading edge is disposed about one hundred and eighty degrees from the second leading edge when the first leg is compressed to the second leg. The first leg has a first channel. The first channel extends through each leading edge of the recesses on the first leg. The second leg has a second channel. The second channel extends through each leading edge of the recesses on the second leg. The first channel is in registration with the second channel when the first leg is compressed with the second leg.

According to another aspect of the present disclosure, the clip has a plurality of first recesses each forming a polygonal shape.

According to yet another aspect of the present disclosure, the clip has the plurality of second recesses each forming a polygonal shape.

According to a further aspect of the present disclosure, the clip has the first channel being unconnected to the second channel.

According to another aspect of the present disclosure, the clip has the first clip leg with a longitudinal axis. The first channel extends a depth from an outer clip surface to the longitudinal axis.

According to another aspect of the present disclosure, the clip has the first channel extending across the first leg and connecting each of the plurality of first recesses.

According to an aspect of the present disclosure, the clip has the first leg with at least one distal most recess and a proximal most recess. The first channel commences at a first distal most leading edge of the distal most recess, and the first channel terminates at another second proximal most leading edge of the proximal most recess.

According to another aspect of the present disclosure, the clip the first channel and the plurality of first recesses each have a depth and collectively forming a tissue exudation structure on the first leg.

According to another aspect of the present disclosure, the clip has the second channel and the plurality of second recesses each with the depth and collectively forming the tissue exudation structure on the second leg.

### BRIEF DESCRIPTION OF THE DRAWINGS

FIG. 1 is a front view of a surgical clip according to the present disclosure;

FIG. 2 is a perspective view of the clip of FIG. 1 having a generally non-limiting “V” shape;

FIG. 3 is a top elevation view of the clip of FIG. 2 showing a number of recesses in a first predetermined gripping pattern on the first leg and a number of recesses in a second predetermined gripping pattern on the second leg;

FIG. 3A is a top elevation close up view of a recess of the clip of FIG. 3;

FIG. 4 is another top elevation view of another embodiment of the clip of FIG. 2 showing a first predetermined gripping pattern on the first leg and a second predetermined gripping pattern on the second leg with a rib therebetween;

FIG. 5 is another top elevation view of another embodiment of the clip of FIG. 4;

FIG. 6 is still another top elevation view of another embodiment of the clip of FIG. 4;

FIG. 7 is yet another top elevation view of another embodiment of the clip;

FIG. 7A shows a cross sectional view of the clip with a recess of the first leg aligned over a recess of the second leg;

FIG. 8 is a close up top view of a shaped recess of the clip;

FIG. 8A is a cross sectional view of the shaped recess of FIG. 8 along line A-A of FIG. 8;

FIG. 9 is a view of the compressed hemostatic clip occluding a vessel;

FIG. 10 is a view of a reverse diamond shape pattern of the recesses of the compressed hemostatic clip; and

FIG. 11 is a cross sectional view of the reverse diamond shape of FIG. 10.

### DETAILED DESCRIPTION OF THE EMBODIMENTS

As used in this specification and the appended claims, the singular forms “a,” “an,” and “the” include plural referents unless the context clearly dictates otherwise. It should be appreciated that these designation form no limitations on the present disclosure whatsoever.

The surgical hemostatic clip of the subject disclosure is applied to body tissue by an appropriate surgical clip applying apparatus. A surgical clip applying apparatus generally has structure to position the clip relative to the tissue to which it will be applied, and a mechanism to deform the clip, usually by compressing the clip at a preselected position thereof to clamp the tissue or vessel.

Some surgical instruments suitable for use in the application of the surgical hemostatic clips described herein are set forth in commonly assigned U.S. Pat. No. 4,509,518 to McGarry et al.; U.S. Pat. Nos. 5,084,057 and 5,100,420 to Green et al.; U.S. Pat. No.: 5,269,792 to Kovac; U.S. Patent Application Ser. No. 60/617,016; U.S. patent application Ser. No.: 11/245,523 filed on Oct. 7, 2005, U.S. Provisional Patent Application Ser. No.: 60/617,104 filed on Oct. 8, 2004 and U.S. Provisional Patent Application Ser. No.: 60/617,016 filed on Oct. 8, 2004, the disclosures of which are all herein incorporated by reference.

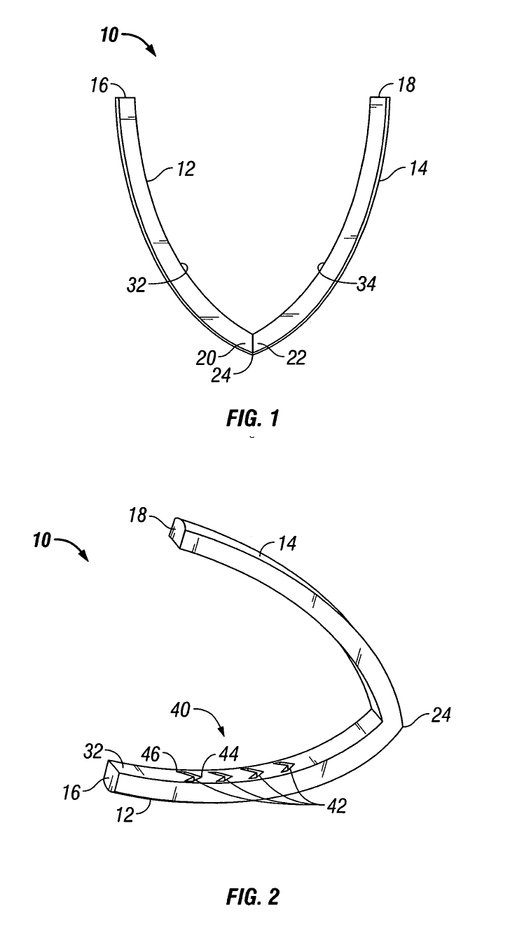

Referring now to FIG. 1, surgical hemostatic clip 10 includes a first leg 12 and a second leg 14. The first leg 12 has a distal end region 16. The second leg 14 has a distal end region 18. Each of the legs 12,14 has respective proximal end regions 20 and 22 that connect the legs 12, 14 to one another. The proximal end regions 20 and 22 of each of the legs 12, 14 of hemostatic clip 10 are connected to one another forming an apex 24. The apex 24 of clip 10 has a generally V-shaped configuration as shown in FIG. 1. One skilled in the art should appreciate that the proximal end regions 20, and 22 may be connected differently to form a “U” shaped clip or another symmetrical or asymmetrical configuration. The clip 10 may have the form of the letter “U”, V, or similar symmetric or asymmetric shapes resembling a broad “U” or V in cross profile or another shape such as one having an occluding portion and a compression portion. The occluding portion is one that can contact the tissue and/or vessel to occlude a vessel. The compression portion has a surface that can receive a compression force from an instrument in order to form the hemostatic clip 10 over the vessel to interrupt fluid flow through the vessel without damaging or harming the tissue or vessel. Various configurations for the compression portion and occluding portion are envisioned and the pattern of the hemostatic clip 10 may be used with various different clip geometries and the present disclosure is not limited to any specific clip geometry. Each of the legs 12 and 14 of hemostatic clip 10 define a respective tissue gripping surface on a lateral side of the hemostatic clip 10. It is envisioned that the desired tissue such as an artery, vessel or vein is clamped between the tissue gripping surfaces between the legs 12, 14 during application of the surgical hemostatic clip 10 for occlusion, or notably the obstruction or a closure of a passageway or vessel. Notably, leg 12 defines tissue gripping surface 32, while leg 14 defines tissue gripping surface 34.

FIG. 2 shows a perspective view of the clip 10 having a “V” shape; however it should be appreciated that instead of the “V” shape of FIG. 2, the clip 10 may have a “U” shape or another clip shape or desired geometry. Referring to FIG. 2, the tissue gripping surface 32 of the clip 10 has a first predetermined gripping pattern 40. As used herein, the term “gripping pattern” means any arrangement, structure or pattern that promotes exudation of tissue. The gripping pattern assists with the compressed clip 10 being retained on the vessel for occlusion purposes. As used herein, the term “tissue exudation” means the process upon which the tissue gradually moves, oozes or traverses into a recess, cavity, lateral surface, apex, distal end, chamfer, textured surface or structure of the clip to remain frictionally engaged thereon such that the clip remains on the desired tissue without damaging the vessel or tissue until physically removed or with degradable clips, until the clip disintegrates. One of ordinary skill in the art should appreciate that the clip 10 and gripping pattern 40 does not pierce tissue, and instead the clip 10 when deformed and in a compressed stated applies a compressive force to the outer surface of tissue in order to provide for occlusion of the tissue or vessel.

The predetermined gripping pattern 40 grasps and retains tissue for occlusion of a vessel in a manner that is particularly advantageous. The tissue is gripped and is retained on the clip 10 when the clip is compressed on tissue. The tissue will remain gripped on the clip 10 for a period of time, or until removed for occlusion during the entire surgical procedure.

In the embodiment shown in FIG. 2, the clip 10 has the first predetermined gripping pattern 40 with a number of recesses 42. Each of the recesses 42 may have a shape or geometry that is particularly advantageous for gripping and retaining tissue. In this embodiment, each recess 42 is generally triangular or “V” shaped and has an apex 44 and base portions 46. In another embodiment, the recesses 42 may be “U” or “C” shaped. In still another embodiment, the recesses 42 may be orthogonal or have a slight curvature. The “V” shaped recesses 42 grip tissue when occluding and does not damage the occluded vessel, artery, vein or passageway. The V-shape of the recess 42 permits tissue exudation during the application of hemostatic clip 10 to tissue during compression of the clip 10. The exudation of tissue into recesses 42 inhibits movement of the hemostatic clip 10 relative to the applied vessel.

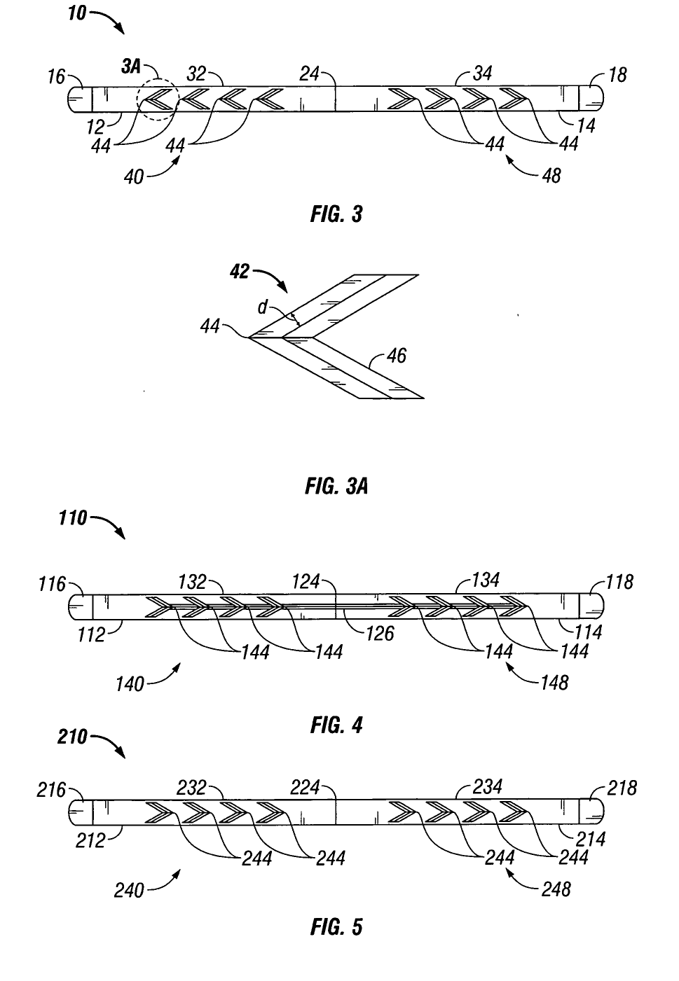

Referring now to FIG. 3, there is shown a top elevation view of the clip 1 0 showing the tissue gripping section 32 of the first leg 12 and the tissue gripping section 34 of the second leg 14. In this embodiment, the tissue gripping section 32 has first predetermined gripping pattern 40. The tissue gripping section 34 of the second leg 14 has a second predetermined gripping pattern 48. It should be appreciated that the first and second gripping patterns 40, 48 may be the same or may be different. Additionally, the tissue gripping sections 32, 34 each may have one or more different patterns thereon.

The first predetermined gripping pattern 40 has a number of “V” shaped recesses 42 in an optimal tissue gripping pattern. As shown, each of the “V” shaped recesses 42 has apex portion 44, the base portions 46, and a slight depth “d” as shown in an enlarged view of FIG. 3A. Referring again to FIG. 3, each apex portion 44 of the tissue gripping surface 32 points towards distal end 16 of the clip 10. Likewise, each apex portion 44 (of tissue gripping surface 34) points toward the distal end 18 of second leg 14. As shown each of the recesses 42 of the first predetermined gripping pattern 40 is in registration with each of the recesses 42 of the second predetermined gripping pattern 48 when the clip 10 has been deformed. Notably, the registration of the recesses 42 is advantageous as the orientation promote an overall or alignment between recesses 42 of the first leg 12 and the second leg 14 and is intended to further optimally inhibit movement of the hemostatic clip 10 relative to the vessel, artery or vein to which it is applied when the first leg 12 is compressed toward second leg 14 to deform clip 10.

As used herein, the term “in registration” or “registering” means that the recesses 42 of the tissue gripping surface 32 form a pattern have a leading edge, sidewall or substructure in the recess 42 that substantially aligns in location along the legs 12, 14 with another pattern of recesses 42 of the tissue gripping surface 34. The alignment promotes the tissue, vessel, or vein to contact the legs 12 and 14. The alignment allows the tissue to exude into the depth “d” of each of the recesses 42 and be frictionally engaged thereon for occlusion until removed. Thus, a superior benefit is achieved as the clip 10 provides that only a minimal (but optimal) amount of tissue gripping surfaces 32, 34 has a gripping feature thereon to promote exudation. This maximizes an amount of friction to retain the tissue on the clip 10. For the purposes of the instant clip 10, the term “in registration” or “in registering” does not necessarily mean that the recesses 42 of leg 12 and the recesses 42 of leg 14 are in an exact mirror image configuration. Instead, the recesses 42 may overlie or overlap one another when leg 12 is compressed to leg 14. Here, the recess 42 of the first clip leg 12 may overlie, or overlap with the recesses 42 of the second clip leg 14 when compressed while not strictly being in an exact mirror image configuration.

Referring now to FIG. 4, there is shown another top elevation view of another alternative embodiment of the clip 110 showing the tissue gripping section 132 of the first leg 112 and the tissue gripping section 1 34 of the second leg 114. In this embodiment, the tissue gripping section 132 has the first predetermined gripping pattern 140 and the tissue gripping section 134 has the second predetermined gripping pattern 148. In this embodiment, the first predetermined gripping pattern 140 has “V” shaped recesses 142. These recesses 142 include apex portions 144 which point toward the apex 124 of the clip 110. The second predetermined gripping pattern 142 has apex portions 144 which points towards distal end 118 of the second leg 114. Further, the clip 110 has a channel 126. The channel 126 extends opposite the apex 124 of the clip 110 or at a complementary location where the first leg 112 intersects the second leg 114. The channel 126 assists with the occlusion of the vessel by forcing the tissue between first leg 112 and second leg 114 to compress around and into the channel 126.

One known surgical clip is U.S. Pat. No. 5,269,792 to Kovac, et al. (hereinafter “Kovac”) which is herein incorporated by reference in its entirety. Kovac discloses at column 10, lines 35 though 37 that the surgical clip has a number of angled grooves extending from one edge of the clip to another edge of the clip. Kovac discloses that the angled grooves improve the grip of the clip on the tissue. The present clip 110 of FIG. 4 is advantageous over Kovac as the recesses 142 of the first clip leg 112 cooperate with the recesses 142 of the second clip leg 114 when the clip 110 is compressed to form a two dimensional polygonal pattern.

Referring now to FIG. 5, there is shown another top elevation view of another alternative embodiment of the clip 210 showing the tissue gripping section 232 of the first leg 212 and the tissue gripping section 234 of the second leg 214. In this embodiment, the tissue gripping section 232 has a first predetermined gripping pattern 240 and the tissue gripping section 234 has a second predetermined gripping pattern 248 with the first predetermined gripping pattern 240 having the apex portion 244. Each apex portion 244 of each recess 242 points towards the apex 224 of clip 210. Likewise, the second predetermined gripping pattern 248 has each apex portion 244 of each recess 242 pointing toward the distal end 218 of the second leg 214. Clip 210 is similar to clip 110 of FIG. 5, but is formed without any channel 126 as shown in FIG. 5. As can be understood, the clip 210 will be positioned over the desired vessel, vein or artery. The clip 210 will then be compressed to occlude the desired vessel, vein or artery using the relevant surgical instrument such as a clip applier.

The recesses 242, on the first clip leg 212 are disposed over and in registration with the recesses 242 of the second clip leg 214. When compressed, the recesses 242 of the first clip leg 212 and the second clip leg 214 together form a two dimensional polygorial or diamond shaped pattern with the tissue being disposed therebetween as illustrated in FIG. 10. The tissue exudes into the recesses 242 of each clip leg 212, 214, and is firmly retained therein. As stated, in this manner, an apex portion 244 of the one recess 242 on the first clip leg 212 is disposed one hundred and eighty degrees from a corresponding apex portion 244 of the second clip leg 214 when clip 210 is compressed.

Referring now to FIG. 10, there is shown a close up view of the clip 210 having the first leg 212 compressed toward the second leg 214. As can be understood from the figure, the leg 212 is compressed toward the leg 214 so the clip 210 can be applied to the desired tissue. Here, the recess 242 of the first clip leg 212 is advantageously disposed over the recesses 242 of the second clip leg 214 when clip 210 is compressed. Recess 242 of leg 214 is shown in phantom broken lines for illustration purposes only. Here, in FIG. 10, the recesses 242 of the first clip leg 212 are in registration with the recesses 242 of the second clip leg 214 when clip 210 is compressed as shown in phantom broken lines.

Together, the recesses 242 of the first clip leg 12 which are disposed over the recesses 242 of the second clip leg 214 forms a two dimensional diamond shaped pattern with the tissue being disposed therebetween as shown in FIG. 10 with the recesses 242 if second leg 214 shown in phantom lines. In this manner, an apex portion 244 of the one recess 242 on the first clip leg 212 is disposed one hundred and eighty degrees from apex portion 244 of the recess 242 of the second clip leg 214 when the clip 210 of FIG. 5 is compressed.

Referring to FIG. 11, there is shown a cross sectional view of the compressed clip 10 of FIGS. 5 and 10 with the first leg 212 compressed over the second leg 214. The compressed clip 210 has the diamond shaped pattern (with the recess 242 of the first clip leg 212 disposed over the recesses 242 of the second clip leg 214). As can be seen from the cross sectional view, the tissue as illustrated by reference letter “T” traverses into the recess 42 of the first clip leg 12 and into the recess 242 of the second clip leg 214 with the apex portion 244 (of the one recess 242 on the first clip leg 212) disposed one hundred and eighty degrees from another apex portion 244 of the recess 242 of the second clip leg 214. The compressed clip 210 has a two dimensional diamond shaped pattern when the recesses 242 to overlap to promote tissue exudation. The compressed clip 210 having a two dimensional diamond shaped pattern when the recesses overlap firmly holds the clip 10 on the tissue to block the flow of fluid therethrough.

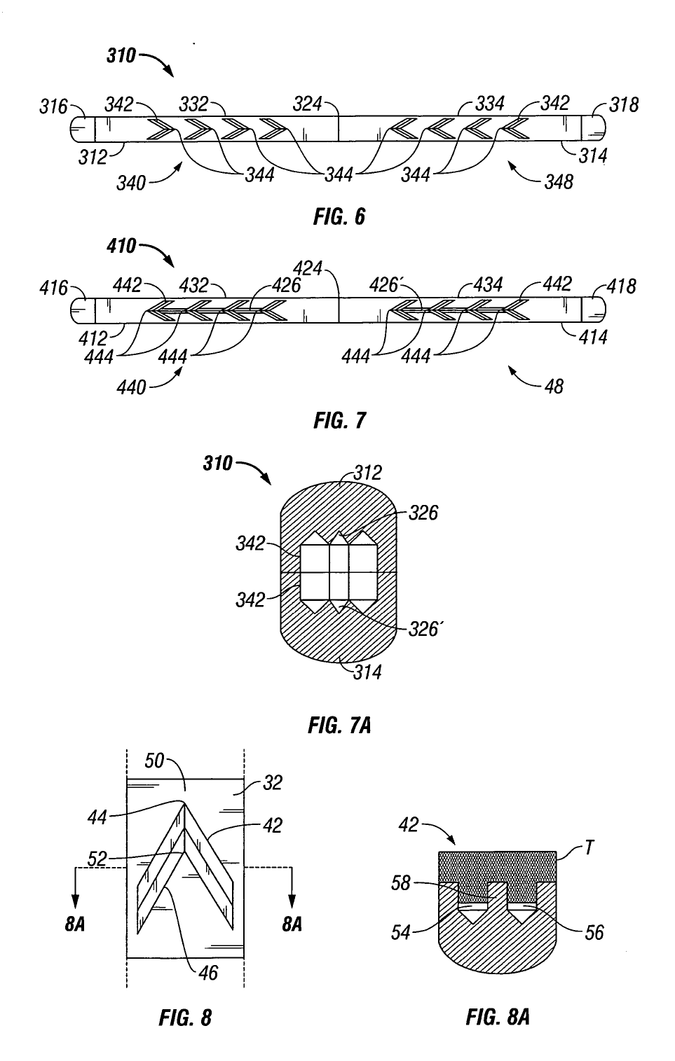

Referring now to FIG. 6, there is shown another top elevation view of another alternative embodiment of the clip 310 showing the tissue gripping section 332 of the first leg 312 and the tissue gripping section 334 of the second leg 314. In this embodiment, the first predetermined gripping pattern 340 has the apex portion 344 of each of the recesses 342 pointing toward the apex 324 of the clip 310. Likewise, the second predetermined gripping pattern 348 has the apex portion 344 of each of the recesses 342 also pointing toward the apex 324 of the clip 310.

Referring now to FIG. 7, there is shown another top elevation view of the embodiment of the clip 410 of FIG. 6 showing the tissue gripping section 432 of the first leg 412 and the tissue gripping section 434 of the second leg 414. Here, the first predetermined gripping pattern 440 is reversed with the apex portion 444 of each of the recesses 442 pointing toward distal end 416 of the first leg 412. Also, the second predetermined gripping pattern 442 is made with each apex portion 444 pointing to the apex 424 of the clip 410. The clip 410 in this embodiment further has the channel 426 that is a segmented channel pattern. The channel 426 on the first leg 412 does not connect with the channel 426′ on the second leg 414. In contrast, the channel 426 connects the apex portion 444 of each recess 442 with one another then terminates. Further, the channel 426′ on the second leg 414 connects each apex portion 444 of each recess 442 with one another. This permits tissue exudation on the recesses 442 and channel 426 of the first clip leg 412 and tissue exudation in the recesses 442 and channel 426′ of the second clip leg 414. Referring now to a cross sectional view of the clip of FIG. 6 showing the first leg 312 compressed to the second leg 314 (FIG. 7A), one can see that an interior space of recess 342 of the first leg 312 substantially align with another recess 342 of the second leg 314. In this manner, the tissue can enter each recess 342 for maximum tissue exudation and to promote occlusion of the desired vessel. In the embodiment shown in FIG. 7, the channel 426 of the first leg 412 also substantially aligns with the channel 426′ on the second leg 414 for promotion of tissue exudation and to assist retaining the clip on the desired tissue.

FIG. 8 shows a close up top view of one recess 42 of the clip 10 of FIGS. 1 through 3. As can be understood, the recess 42 is separated from another adjacent recess by a distance. The leg 12 has an outer surface 50 that is generally flat. Each of the recesses 42 has a “V” shape. Each recess 42 is made with an apex portion 44. Closely adjacent to the apex portion 44 of the recess 42 is a leading edge 52. The leading edge 52 is pointed to a tip and in this embodiment assists with gripping the exuded tissue that enters the recess 42 when the clip 10 is compressed. This recess 42 is disposed on the tissue gripping surface 32 of the first leg 12; however the recess 42 may be disposed in other locations. In this embodiment, the leading edge 52 is pointed in a direction parallel to a lateral edge of the clip 10, however, the leading edge 52 may be formed so as to point in other directions angled from the lateral edge of the clip 10 or even be formed perpendicular to the lateral edge of the clip 10 or even at a number of intermediate angles therebetween depending on the recesses 42 of the other leg. Various configurations are possible and one skilled in the art should appreciate that the clip 10 may be formed with the leading edge 52 pointing in a number of different locations so long as the recesses 42 of one leg 12 are in registration with recesses 42 of another leg 14 to form the two dimensional polygonal or diamond shaped pattern as shown in FIG. 10. Various configurations are possible.

Referring to a cross sectional view of one recess 42 as shown in FIG. 8A along line A-A of FIG. 8, the recess 42 has a first portion 58 and a first and second sub recess 54, 56. The first portion 58 splits the first and second sub recesses 54, 56. When the clip 10 is compressed, the relevant tissue section shown as reference letter “t” will ooze, exude or otherwise slightly traverse into the first and second sub recesses 54, 56 of the recess 42 and be retained therein by the leading edge 52 and by a frictional engagement of the lateral side walls of the recess 42. In another embodiment, the lateral side walls of the recess 42 may further optionally overhang over the first and second sub recesses 54, 56 to assist with tissue exudation. Likewise, in another embodiment, the first portion 58 of the leg 12 may further have a surface treatment to further assist with clip retention.

With reference to FIG. 1, the surgical hemostatic clip 10 of the subject disclosure may be of any dimension suitable for application to vessels and body tissue. In one embodiment, the length of the clip 10 is about 7.95 millimeters and the width of the clip 10 from an outer surface of the first leg 12 to an outer surface of the second leg 14 is about 4.57 millimeters, and the surgical clip 10 is intended to be used with a five millimeter trocar. One of ordinary skill in the art will recognize that other dimensions can also be used, and the dimensions of the clip 10 may be modified to various other dimensions to fit various clip appliers, trocars, tissue, vessels, arteries or other surgical procedures.

The structures described herein may be formed in clip 10 by molding or by applying an appropriate stamping force to the faces of legs 12 and 14. Alternatively, structures may be formed by machining clip 10 or by other known metal or polymer processing techniques. For example, clip 10 may be molded with the structures formed therein. The surgical hemostatic clip 10 of the present disclosure may be fabricated from any biocompatible material including stainless steel, titanium, and tantalum, as well as plastic materials including biocompatible polymers, or a combination of materials thereof.

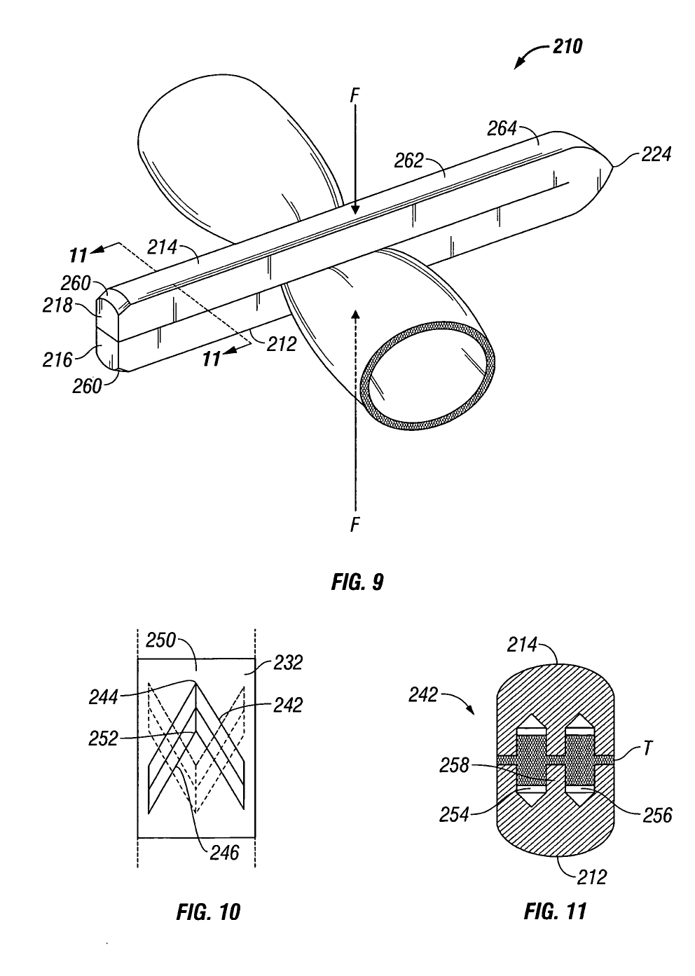

Referring now to FIG. 9, there is shown the clip 10 of the embodiment of FIGS. 1 through 3 in a compressed state and over a vessel such as an artery or vein for occlusion. One skilled in the art should appreciate that no fluid traverses through the vessel when the clip 10 is compressed over the vessel. Notably, the first and the second predetermined gripping patterns 40, 48 grip and retain an amount of tissue therein and retain the clip 10 on the vessel for the entire surgical procedure or as desired until physically removed.

FIG. 9 shows a view of the first distal end 16 and the second distal end 18 of the legs 12,14. Each of the ends has a chamfer 60. The chamfer 60 is on an outer surface of distal ends 16, 18 of the clip 10. The clip 10 further may have a textured feature 62. The textured feature 62 is on an outer surface 64 of the clip 10. Textured feature 62 assists with retaining the clip 10 in the clip applier 10. Textured feature 62 may be a surface roughness, a number of striations, a number of protuberances, a rough or grainy surface quality or any other surface quality that increases a coefficient of friction to the outer surface 62, and assists with manipulating the clip 10, and compressing the clip 10 to apply the clip 10 to body tissue.

Referring to FIG. 9, the method of making the clip 10 provides that the clip 10 has an improved surface treatment. The method 70 has the following steps. First, the clip 10 is stamped, forged or molded and formed into a generally desired shape as disclosed in FIGS. 1 through 11. Various shapes are possible and within the scope of the present disclosure. Accordingly, at step 72, the clip 10 is stamped, forged, molded, or machined into the desired “V” or “U” shape. The clip 10 is a metallic material.

In one embodiment, the clip 10 is an ASTM F67 grade 1 Titanium. However, the clip 10 may be other materials or in another embodiment may be an alloy, steel, metal, another grade of titanium, or another similar biocompatible or suitable implantable material. Thereafter, the method has the step of heating the clip 10. The clip 10 is, in one embodiment, heated in a vacuum. The method also has the step of slow cooling the clip 10 to strengthen and harden the clip 10 to change a material characteristic of the clip 10. The cooling of the clip 10 is performed in order to stress relieve and change the clip hardness and Rockwell strength of the clip 10. Various cooling parameters in order to modulate the Rockwell strength of the clip 10 are possible and within the scope of the present disclosure.

At step 74, the clip 10 is heated to a desired temperature. The desired temperature is in one embodiment 1,275 degrees Fahrenheit for a predetermined time period. In one embodiment, the time period may be one hour, however other suitable time periods are possible. Alternatively, the desired temperature is in another embodiment 1,250 degrees Fahrenheit. However, this desired temperature is non-limiting and any acceptable temperature may used in order to heat the clip to a temperature where annealing is possible depending on the material of the clip 10. For the purposes of the temperature range given above the clip 10 is made from titanium. The clip 10 is heated in a suitable furnace, oven or other suitable device or heating apparatus. Thereafter, the method 70 proceeds to step 76. The heated clip is then soaked for a period of time. The clip 10 is soaked in an inert gaseous substance. In one embodiment, the clip 10 is soaked in an inert Argon gas for a predetermined amount of time or in another similar acceptable soaking substance. Thereafter, the method 70 proceeds to step 78. At step 78, the heated clip 10 contacts an argon gas. The clip 10 is first placed and heated in a suitable compartment. After the clip is heated, the clip is then soaked with the Argon gas. The argon gas controls and actively modulates or restructures the crystal size and alignment of the titanium of the clip in proportion to the argon gas as the titanium cools in order to control the microstructure of the clip 10. The heated clip 10 is cooled at a uniform rate. In one embodiment, the clip 10 may be furnace or air cooled.

Thereafter, the method 70 proceeds to step 80. At step 80, the clip is removed from the compartment and tested for hydrogen content. The analysis is nondestructive. The analysis measures the sample and the results are independent of the specific chemical form of the hydrogen or related hydrogen compounds present. At step 80, the clip 10 is tested using a cold neutron prompt gamma ray activation analysis apparatus (CNPGAA) or similar machine. Alternatively, the clip 10 may be tested using an X-ray diffraction device. Thereafter, the method further has the steps of machining a first plurality of recesses 42 with each having a first apex portion in a first leg 12 of the clip 10, and machining a second plurality of recesses 42 having a second apex portion in a second leg 14 of the clip. When the clip is compressed the second plurality of recesses are in registration with the first plurality of recesses. The first apex portion is disposed one hundred and eighty degrees from the second apex portion to form when compressed a two dimensional diamond shaped pattern as shown in FIG. 10. Thereafter, the method also has a tumbling process. The tumbling process involves placing the clips in another sealed tumbling compartment, and moving the compartment to impart kinetic energy to the clips for a predetermined period of time. The tumbling compartment may have another rigid resilient substance therein. The clips contact an inner surface of the tumbling compartment or the substance. The repeated contact gives the clip one or more holding surfaces to promote retention on tissue. Further, the repeated contact gives an exterior of the clips 10 a predetermined textured finish conducive for surgery.

It is to be understood that the above described embodiments are only illustrative of the application of the principles of the present disclosure. Numerous modifications and alternative arrangements may be devised by those skilled in the art without departing from the spirit and scope of the present disclosure. The appended claims are intended to cover such modifications and arrangements.

What is claimed is:

1: A surgical clip for a clip applier, the surgical clip comprising:
a first leg connected to a second leg at a first location;
wherein the first leg is separated from the second leg by a predetermined distance at a second location spaced from the first location, the first leg forming a first distal end and the second leg forming a second distal end, the first leg having a tissue gripping region and the second leg having a second tissue gripping region;
wherein the first tissue griping region has a first recess on the first leg, and the second tissue gripping region has a second recess on the second leg; and
wherein when the clip is compressed the first recess is compressed towards the second recess, wherein the first recess and the second recess overlap to form a two dimensional polygonal pattern with one another.

2: The surgical clip of claim 1, wherein the first leg has a plurality of first recesses and the second leg has a plurality of second recesses having substantially the same orientation, each of the first recesses registering with another of the second recesses when the first clip leg is compressed toward the second clip leg.

3: The surgical clip of claim 1, wherein when the first recess is compressed toward the second recess, the first and the second recess forms a two dimensional diamond shaped pattern.

4: The surgical clip of claim 1, wherein the first recess has an apex portion and a first and a second distal leg portion extending from the apex portion

5: The surgical clip of claim 4, wherein the second recess has an apex portion and a first and a second distal leg extending from the apex portion.

6: The surgical clip of claim 5, wherein when the first clip leg is compressed to the second clip leg, the apex portion of the first clip leg and the apex portion of the second leg point toward opposite directions.

7: The surgical clip of claim 2, wherein the first leg has the plurality of first recesses;
wherein each of the first recesses have an apex portion and a first and a second leg extending from the apex portion with the apex portions of each of the recesses pointing toward the same direction.

8: The surgical clip of claim 7, wherein the second leg has the plurality of second recesses with each of the recesses having an apex portion and a first and a second leg extending from the apex portion; and
wherein the apex portions of the second recesses point toward an opposite direction relative to the apex portion of the first recesses.

9: The clip of claim 1, wherein each recess is a substantially “V” shaped recess.

10: The surgical clip of claim 2, further comprising a longitudinal channel, the longitudinal channel extending through each of the recesses of at least the first leg and the second leg.

11: The surgical clip of claim 1, further including a grip feature on an outer clip leg surface.

12: The surgical clip of claim 2, wherein each of the recesses do not contact another of the recesses.

13: The surgical clip of claim 2, wherein the surgical clip is a hemostatic clip and is made from a material selected from the group consisting of stainless steel, a polymer, titanium, a biocompatible material, and any combinations thereof.

14: A surgical clip for a clip applier, the surgical clip comprising:
a first leg connected to a second leg at a first location;
wherein the first leg is separated from the second leg by a predetermined distance at a second location spaced from the first location, the first leg having a first distal end and the second leg having a second distal end;
a first gripping pattern on the first leg having a plurality of first recesses;
a second gripping pattern on the second leg having a plurality of second recesses;
wherein the plurality of first recesses are in registration with the plurality of second recesses when the first leg is compressed towards the second leg such that each of the first recesses of the first plurality of first recesses registers with a respective second recess of the second plurality of recesses to form a two dimensional polygonal pattern;
wherein the first leg has a channel extending through each of the recesses on the first leg; and
wherein the second leg has a second channel extending through each of the recesses on the second leg.

15: The surgical clip of claim 14, wherein each recess is “V” shaped.

16: The surgical clip of claim 14, wherein both the first channel and the second channel do not connect with one another.

17: The surgical clip of claim 14, wherein the first channel and the second channel are connected.

18: The surgical clip of claim 14, wherein each recess is “U” shaped.

19: The surgical clip of claim 14, wherein when each of the first recesses overlap and align with the second recesses when the clip is compressed, the first recesses and the second recesses together forming a two dimensional diamond shaped pattern with one another

20: A surgical clip for a clip applier, the surgical clip comprising:
a first leg connected to a second leg at a first location;
wherein the first leg is separated from the second leg by a predetermined distance at a second location spaced from the first location, the first leg having a first distal end and the second leg having a second distal end, the first leg having a tissue gripping region and the second leg having the tissue gripping region;
wherein the first tissue griping region has a plurality of first “V” shaped recesses on the first leg with each recess having a first apex;
wherein the second tissue gripping region has a plurality of second “V” shaped recess on the second leg with each recess having a second apex; and
wherein when the clip is compressed the first “V” shaped recesses on the first leg align and overlap with the second “V” shaped recesses on the second leg; and
wherein when the first recess is compressed to the second recess, each apex of the first recesses and each apex of the second recesses points in an opposite direction.
